(19) United States
(12) Plant Patent Application Publication
(10) Pub. No.: US 2017/0142880 P1
(43) Pub. Date: May 18, 2017

(54) Cherry tree named 'PA7UNIBO'

(50) ;

(71) Applicant: Alma Mater Studiorum-Università Di Bologna, Bologna (IT)

(72) Inventors: Stefano LUGLI, Bologna (IT); Riccardo CORREALE, Bologna (IT); Michelangelo GRANDI, Bologna (IT)

(21) Appl. No.: 15/330,889

(22) Filed: Nov. 14, 2016

(30) Foreign Application Priority Data
Nov. 12, 2015 (EP) 2015/2594

(51) Int. Cl. A01H 5/08

(52) U.S. Cl. PLT/181

(57) ABSTRACT

Variety Denomination ‘PA7UNIBO’ is a novel cherry tree derived from a seedling of unknown cross which was planted at Vignola, Modena Province, Italy. The genus and species (cultivar) of ‘PA7UNIBO’ is Prunus avium. It was initially propagated by grafting to root stocks of varying vigor and, after testing in districts and plantings of differing types, proved to have qualities for a promising new cultivar for the market.

### CROSS-REFERENCE TO RELATED APPLICATIONS

This application claims priority under 35 U.S.C. §119 to CPVO (Community Plant Variety Office) Application No. 201502594, titled, ‘PA7UNIBO’, which was filed on Nov. 12, 2015, which is hereby incorporated herein by reference in its entirety.

The European Trademark for ‘Sweet Stephany’ was registered on Aug. 1, 2016 as Registration No. 014997911, in the name of Alma Mater Studiorum—Universita di Bologna. This European Trademark is associated to ‘PA7UNIBO’, and used to distinguish such cherry variety from others.

Latin name and variety denomination: Latin name of the genus/species of the plant claimed (cultivar): Prunus avium (L.)L.

Variety denomination: ‘PA7UNIBO’.

### BACKGROUND OF INVENTION

Prunus avium ‘PA7UNIBO’, originally derived from seedling of unknown cross and called Selection DCA BO B5 A89, was planted at Vignola, Modena Province, Italy, in 2002, and was first cropped in 2004. It was initially propagated by grafting to root stocks of varying vigor, tested in different growing districts and planting densities, and proved to have the properties proper to a promising new cultivar for the market. It picks in the second/third decade of June (or third/fourth week of June) in Modena Province, more precisely, about 27-28 days after ‘Burlat’ and approximately 6-9 days after ‘Bing’.

### BRIEF SUMMARY OF INVENTION

The parentage of the variety is unknown. The fruit is comparable in size to ‘Duroni 3’ and ‘Sunburst’ varieties, and the prevailing caliber is 30-32 mm (definable as very large size). ‘PA7UNIBO’ was selected for its high qualities, including tree growth and yield performance, fruit appearance (medium red, comparable to ‘Burlat’, ‘Kordia’, ‘Lapins’), with excellent taste-flavor properties, aromatic and very sweet with a good level of acidity, instead the color of flesh id medium red (comparable to ‘Germesdorfi 45’, ‘Hedelfinger Riesenkirsche’). The fruit is considered firm. The distinctive traits of ‘PA7UNIBO’ also include mid-to-late picking date, uniform ripening, and high field performance.

The date of cross was in the year 2000. The location of the discovery was Vignola, Modena Province, Italy. The date of the first asexual propagation was in 2008 in Vignola, Modena Province, Italy. ‘PA7UNIBO’ resulted from a cross of unknown varieties, but was initially propagated on root stock of ‘Gisela 6’ in the trial fields of Bologna University to test field and cropping performance in a medium-to-high density planting.

### BRIEF DESCRIPTION OF THE FIGURES

The accompanying photographs show typical specimens of the new variety as depicted in color as nearly true as is reasonably possible in color illustrations of this character.

FIG. 1 illustrates trees of PA7UNIBO at blooming time.

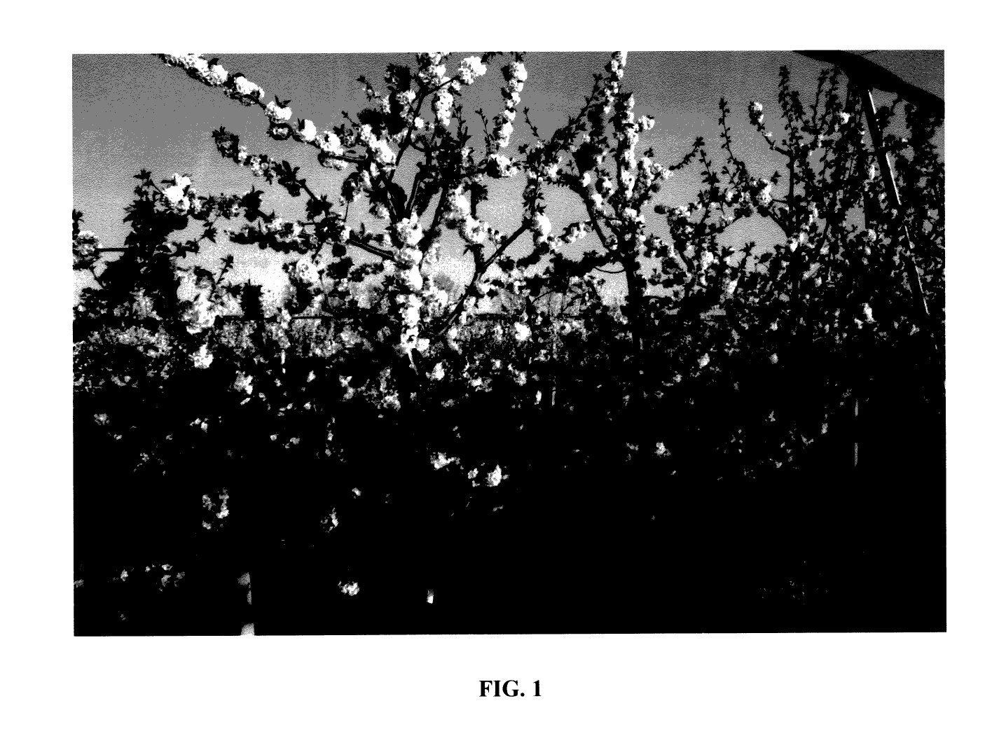

FIG. 2 shows the flowers of PA7UNIBO.

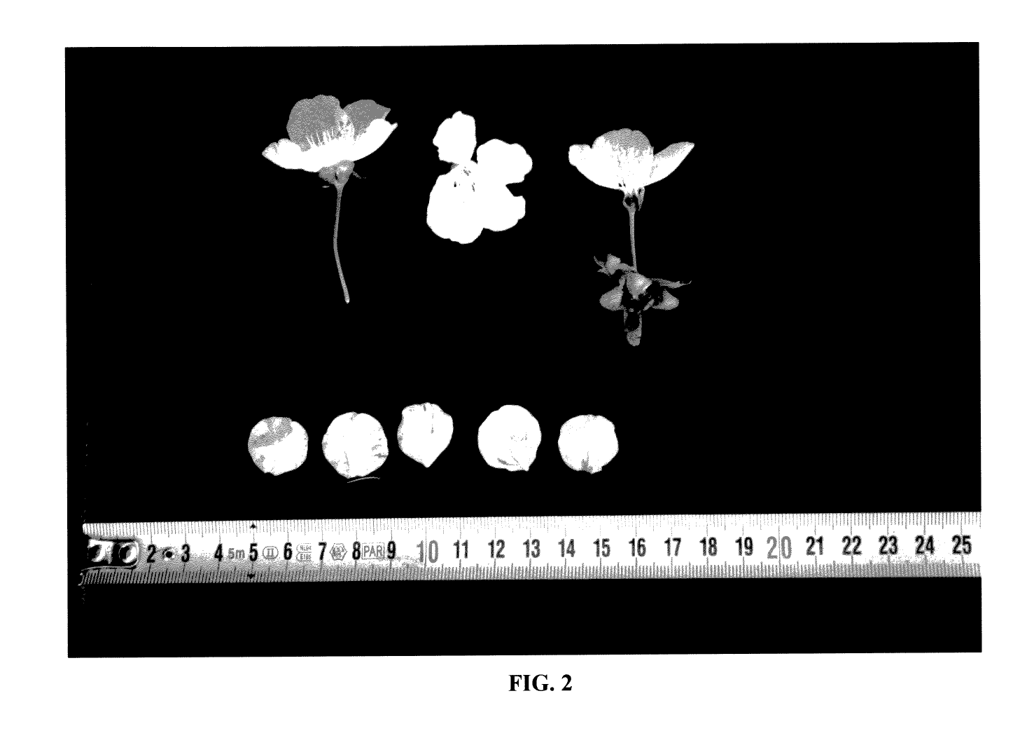

FIG. 3 shows the leaf of PA7UNIBO.

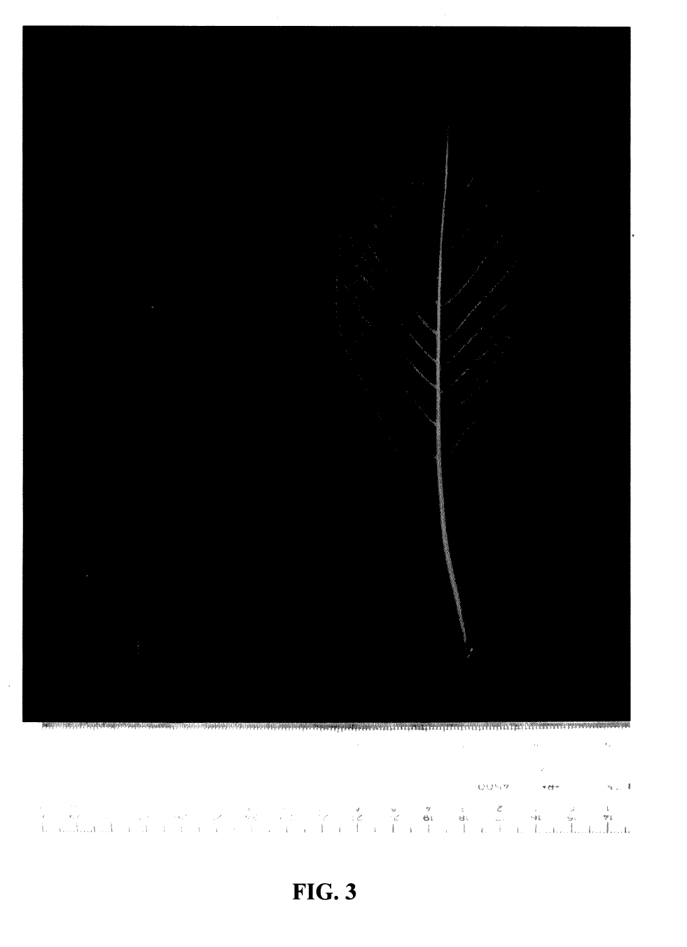

FIG. 4 shows fruits of PA7UNIBO.

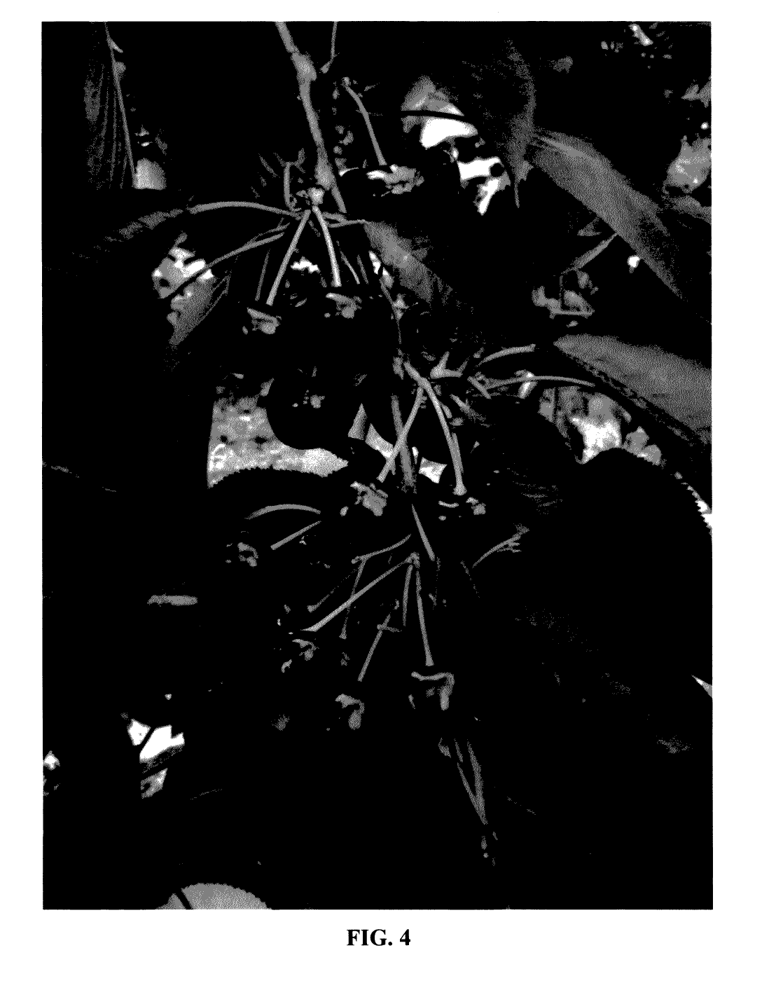

FIG. 5 shows fruits of PA7UNIBO.

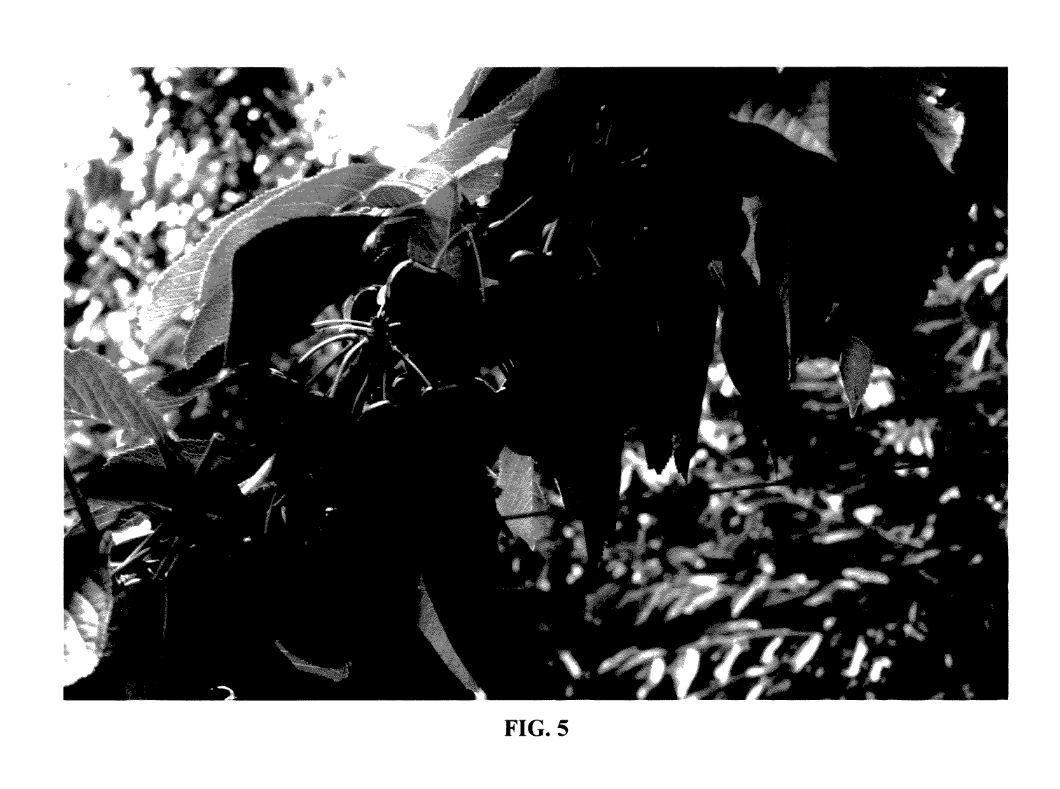

### DETAILED BOTANICAL DESCRIPTION OF ‘PA7UNIBO’

General:
   Age of described tree(s).—5 years slender spindle training system.
   Location of described trees(s).—Vignola, Modena Province, Italy.
   Name of color chart used.—Royal Horticultural Society Colour Chart.
   Name of root stock.—‘Gisela 6’.
Tree:
   Vigor.—High.
   Height.—4.5 meters.
   Spread.—3 meters.
   Growth habit.—Open.
   Canopy density.—Medium.
   Trunk diameter at specified height.—19-22 cm, as measured at about 5 cm above graft joint.
   Trunk color.—gray (201A).
   Bark texture.—Medium rough.
   Tendency toward alternate bearing.—No.
   Winter hardiness.—Unknown; no winter cold damage found in this Po Valley district.
   Chilling requirement.—Unknown.
Branch:
   Length.—150-200 cm.
   Diameter.—3-4 cm.
   Crotch angle.—45° -60° depending on training system.
   Bark color.—reddish-brown (183B) with gray (201A).
   Bark texture.—Medium rough.
Current year shoot:
   Length.—50-60 cm.
   Color.—Reddish-brown (200C/) with fairly widespread gray overtone (201A).
Winter hardiness:
   USDA hardiness zone.—Unknown.
   Chilling requirement.—Unknown.
Flower buds:
   Number per spur.—5-6, but usually 6.
   Shape.—Sub-globose.
   Length.—About 1 cm.
   Diameter.—Under 1 cm.
   Color.—Reddish perules (187C).
Flowers:
   Number per cluster.—Minimum 18, maximum 20 per spur; 3-4 per bud. Bloom is abundant.
   Relative position of petals.—Petals overlap slightly.
   Diameter.—30-35 mm.
   Depth.—10-15 mm, measured as distance from receptacle to maximum petal aperture.
   Is pollen produced?.—Yes.
   Coloration of pollen.—Pale yellow (12A).
Sepal:
   Length.—5-6 mm.
   Shape.—pointed.
   Width.—3-4 mm.
   Apex.—Pointed.
   Base.—Flat.
   Margin descriptors.—Regular margin.
   Color.—Upper surface — Pale green 142B.
   Color.—lower surface — Pale green 142A with red (173A).
Petal:
   Number per flower.—5.
   Shape.—Circular.
   Length.—18-20 mm.
   Width.—16 18 mm.
   Apex.—Slightly sunken.
   Base.—Slightly pointed.
   Margin.—Slightly undulated.
   Color.—Upper surface — White 20D.
   Color.—Lower surface — White 20D.
Bloom:
   Date of bud burst.—Apr. 7-8, 2013.
   Date of first bloom.—Apr. 13, 2013.
   Date of full bloom.—Apr. 16, 2013.
Pedicel:
   Length.—28-35 mm.
   Diameter.—About 1.2 mm.
   Color.—Pale green 144A.
Pistil:
   Quantity.—1.
   Size.—19-21 mm.
   Color.—Pale green 144D.
Anthers:
   Quantity.—28-30, with about 70% of 10-13 mm in length, positioned slightly below the top of the stigma, and the remaining 30% varying from 5-8 mm. White filament (20D).
   Size.—About 1 mm diameter.
   Color.—Orange-yellow 15C.
Stigma:
   Quantity.—1.
   Size.—1.0-1.2 mm diameter.
   Color.—yellow-green (144B).
Styles:
   Quantity.—1.
   Size.—14-16 mm length.
   Color.—Yellow-green (144B).
Ovary/ies:
   Quantity.—1.
   Size.—3-4 mm.
   Color.—Pale green 144A.

Leaves:
Length.—12.2-14.1 mm.
Width.—6.5-6.9 mm.
Shape.—Lanceolate-elliptical.
Apex shape.—Pointed.
Base shape.—Pointed.
Margin.—Fine toothed.
Color.—Upper surface — Dark green 137 B.
Color.—Lower surface — Pale green 147B veined green-yellow (152A/152B) in center.

Petiole:
Length.—4.5-5 cm.
Diameter.—About 2 mm.
Color.—Pale green lower 144A and reddish upper (183A).
Fruit:
   Shape.—Depressed heart-shaped.
   Diameter.—Mostly 30 mm.
   Height.—24-27 mm.
   Weight.—12-13 grams on average.
   Number per cluster.—5-10 per spur.
   Skin color (include ground color and over color).—Shiny dark red (187B) tending to blackish-dark red (187A) at advanced maturity.
   Presence of lenticels.—Yes.
   Density of lenticels.—Medium-high.
   Skin thickness.—Medium-thin.
   Skin texture.—Medium.
   Anthocyanin coloration present near skin?.—No.
   Tenacity of flesh to stone.—Low.
   Flesh color.—Red (53A).
   Flesh texture.—Fine.
   Juiciness.—High.
   Juice analysis (brix, acidity, tss, etc.).—21.9 Brix and 8.6 11.8 g/l malic acid (2012-2015 data).
   Stone size.—Medium-small.
   Stone shape.—Elliptical.
   Stone color.—Pale brown (165D).
Fruit stem:
   Length.—35-38 mm.
   Diameter.—Just over 1 mm.
   Color.—Green (143A).
Harvest:
   Maturity date range.—June 10-June 25, approximately 26 days after ‘Burlat’ and about 6 to 9 days after ‘Bing’. Optimum harvest dates of ‘PA7UNIBO’ in Modena Province are June 15-20.
   Harvest date range (if different).—Same as above. The diploid cultivar ‘PA7UNIBO’ is self-fertile, with the allelic profile of S3S4. Fruit load and yield is high and constant, particularly in the presence of flying insects (e.g., bees, wasps, hornets, beetles, butterflies, etc.). The method of propagation of the variety is represented by budding or grafting. Under observation during planting, growing and harvesting under normal cultural and growing conditions in Modena Province, Italy, ‘PA7UNIBO’ has been tested against the following viruses: ACLSV, ApMV, PRNSV, PDV, CLRV, RpRSV, CNRMV, PPV, TBSV, LChV-1, LChV-1, SLRSV, ArMV, APLPV. The results were negative, i.e., ‘PA7UNIBO’ was free from the above-mentioned viruses. Because of the outstanding and unique horticultural characters of the claimed plant, especially its long harvest period and large size, this cherry can be planted to extend the cultivar's season. Although this new variety of cherry possesses the above-described characteristics, it is to be understood dial variations and fluctuations may occur in the magnitude and qualities due to changes in growing and climate conditions, irrigation, fertilization, pruning and pest control.

What is claimed:

1. A new and distinct cherry tree, as herein described and illustrated.
